(19) United States
(12) Patent Application Publication
(10) Pub. No.: US 2025/0177001 A1
(43) Pub. Date: Jun. 5, 2025

(54) Safety Mechanism

(71) Applicant: Bard Access Systems, Inc., Salt Lake City, UT (US)

(72) Inventor: Daniel B. Blanchard, Bountiful, UT (US)

(21) Appl. No.: 19/052,150

(22) Filed: Feb. 12, 2025

Related U.S. Application Data
(63) Continuation of application No. 17/378,304, filed on Jul. 16, 2021, now Pat. No. 12,226,123.
(60) Provisional application No. 63/053,356, filed on Jul. 17, 2020.

(51) Int. Cl. A61B 17/34

(52) U.S. Cl. CPC A61B 17/3472; A61B 17/3423; A61B 17/3496; A61B 2017/347

(57) ABSTRACT

A safety mechanism for an obturator having a sharp distal tip and a notch proximal to the sharp distal tip. The safety mechanism includes a sheath and clip combination configured to releasably couple to a needle hub. The clip is configured to transition between a coupled configuration and an uncoupled configuration. The clip can include a first arm including a first keyhole aperture and a second arm including a second keyhole aperture. The clip can further include a biasing member connecting the first arm to the second arm, the biasing member configured to bias the clip toward the uncoupled configuration. The first keyhole aperture and the second keyhole aperture are configured to slidingly receive the obturator. The clip can transition from the coupled configuration to the uncoupled configuration when the first keyhole aperture and the second keyhole aperture engage the notch.

### PRIORITY

This application is a continuation of U.S. patent application Ser. No. 17/378,304, filed Jul. 16, 2021, now U.S. Pat. No. 12,226,123, which claims the benefit of priority to U.S. Provisional Application No. 63/053,356, filed Jul. 17, 2020, each of which is incorporated by reference in its entirety into this application.

### SUMMARY

Intraosseous access systems can be configured to penetrate the skin, underlying tissues, and bone cortex to access the medullary cavity of the bone and provide fluid communication therewith. Such intraosseous access systems use an obturator disposed within a needle lumen to prevent tissue or bone fragments from entering the needle lumen and obstructing the fluid pathway. Once the needle has accessed the medullary cavity, the obturator can be removed from the needle assembly. Removal of the obturator raises the risk of accidental stick injuries by the obturator tip. A safety device can be used to significantly reduce the risk of accidental stick injuries. Current safety mechanisms have multiple components that require precise machining leading to increased manufacturing complexity and associated costs. Further, the drag or activation forces can be easily imbalanced leading to failure of the device.

What is needed therefore, is a safety mechanism that has a simplified construction reducing manufacturing complexity and associated costs, improved deployment reliability, and provide a more secure engagement between the obturator tip and a protective sheath. Embodiments disclosed herein include a safety mechanism and associated methods thereof to address the foregoing.

Disclosed herein is a safety mechanism including, an elongate medical device having a shaft extending along a longitudinal axis and defining a first outer diameter, the shaft including a notch extending annularly and defining a second outer diameter, less than the first outer diameter, a sheath including a body defining a sheath lumen configured to receive the elongate medical device therethrough, and a shroud extending axially and including a first sheath aperture and a second sheath aperture, and a clip configured to transition between a first configuration and a second configuration, the clip having a first arm, including a first tab, a first plate, and a first grip, the first tab including a first keyhole aperture, a second arm, including a second tab, a second plate, and a second grip, the second tab including a second keyhole aperture, and a biasing member extending from the first arm to the second arm and configured to bias the clip towards the second configuration, the elongate medical device extending through first keyhole aperture and the second keyhole aperture, the first grip extending through the first sheath aperture, and the second grip extending through the second sheath aperture.

In some embodiments, the first arm and the second arm are spaced in a radially outward position in the second configuration relative to the first configuration. In some embodiments, the first keyhole aperture includes a first lock aperture communicating with a first unlock aperture, and the second keyhole aperture includes a second lock aperture communicating with a second unlock aperture, one or both of the first lock aperture and the second lock aperture having a first inner diameter, and one or both of the first unlock aperture and the second unlock aperture having a second inner diameter larger than the first inner diameter. In some embodiments, the first inner diameter is less than the first outer diameter and equal to or larger than the second outer diameter, and the second inner diameter is equal to or larger than the first outer diameter.

In some embodiments, the first lock aperture and the second lock aperture are axially aligned when the clip is in the second configuration, and the first unlock aperture and the second unlock aperture are axially aligned when the clip is in the first configuration. In some embodiments, the first unlock aperture and the second unlock aperture are spaced radially outward relative to a central axial position in the second configuration. In some embodiments, the first lock aperture is disposed opposite the longitudinal axis from the second lock aperture in the first configuration. In some embodiments, the sheath body is configured to engage a recess of a needle hub, the shroud extends over an outer surface of the needle hub, one or both of the first grip and the second grip engaging an outer surface of the needle hub when the clip is in the first configuration.

In some embodiments, the first grip extends through first sheath aperture to engage a first grip recess disposed in an outer surface of the needle hub, and the second grip extends through second sheath aperture to engage a second grip recess disposed in an outer surface of the needle hub. In some embodiments, one or both of the first grip and the second grip is configured to disengage the needle hub in the second configuration. In some embodiments, the first grip engages the first sheath aperture and the second grip engages the second sheath aperture in both the first configuration and the second configuration.

In some embodiments, the sheath is in a longitudinally fixed position relative the elongate medical device and a distal tip of the sheath body extends distally of a distal tip of the elongate medical device when the clip is in the second configuration. In some embodiments, the first tab, the second tab, the first grip and the second grip extend perpendicular to the longitudinal axis, and the first plate and the second plate extend parallel to the longitudinal axis. In some embodiments, the clip is formed as a monolithic piece from one of a plastic, polymer, metal, alloy, or composite. In some embodiments, the elongate medical device includes one of an obturator, needle, cannula, trocar, or a stylet.

Also disclosed is a method of engaging a safety mechanism with a tip of an elongate medical device including, withdrawing the elongate medical device proximally along a longitudinal axis, through a first keyhole aperture disposed in a first tab of a clip, transitioning the clip from a first configuration to a second configuration, the clip fixedly engaging the elongate medical device in the second configuration to prevent any longitudinal movement therebetween, the clip coupled to a sheath, and disengaging a first grip from a needle hub to disengage the sheath from the needle hub, the first grip coupled to the first tab.

In some embodiments, the clip includes a biasing member configured to bias the clip towards the second configuration. In some embodiments, the method further includes sliding the first tab perpendicular to the longitudinal axis the elongate medical device extending through a first unlock aperture of the first keyhole aperture when the clip is in a first configuration, and extending through the lock aperture when the clip is in a second configuration. In some embodiments, the method further includes engaging the first lock aperture with a notch of the elongate medical device, the inner diameter of the first lock aperture being less than a first outer diameter of the elongate medical device and larger than a second inner diameter of the notch.

In some embodiments, the method further includes fixedly engaging a sheath, coupled to the clip, relative to the elongate medical device, a distal tip of the sheath extending distally of a distal tip of the elongate medical device. In some embodiments, the first grip engages a grip recess disposed in an outer surface of the needle hub when the clip is in the first configuration. In some embodiments, the sheath includes a body defining a sheath lumen configured to receive the elongate medical device there through, and a shroud extending axially and configured to engage an outer surface of the needle hub, the first grip slidably engaged with a sheath aperture disposed in the shroud in both the first configuration and the second configuration. In some embodiments, the clip is formed as a monolithic piece from one of a plastic, polymer, metal, alloy, or composite. In some embodiments, the elongate medical device includes one of an obturator, needle, cannula, trocar, or a stylet.

Also disclosed is a method of manufacturing a safety assembly including, forming a clip from a first material, the clip having a first arm, including a first tab, a first plate, and a first grip, the first tab including a first keyhole aperture, a second arm, including a second tab, a second plate, and a second grip, the second tab including a second keyhole aperture, and a biasing member extending from the first arm to the second arm, folding the first tab and the first grip through an angle of 90 degrees relative to the first plate, folding the second tab and the second grip through an angle of 90 degrees relative to the second plate, forming a sheath from a second material, the sheath including a body defining a lumen and a shroud including a sheath aperture extending therethrough, and coupling the clip to the sheath wherein one of the first grip or the second grip extends through the sheath aperture.

In some embodiments, the method further includes elastically deforming the elongate biasing member such that the first keyhole aperture axially aligns with the second keyhole aperture. In some embodiments, the first material is a sheet of metal and the second material is a polymer.

Also disclosed is a safety mechanism including, an elongate medical device having a shaft extending along a longitudinal axis and defining a first outer diameter, the shaft including a notch extending annularly and defining a second outer diameter, less than the first outer diameter, a sheath including a body defining a lumen configured to receive the elongate medical device therethrough, and a shroud extending axially and including a sheath aperture, and a clip configured to transition between a first configuration and second configuration, the clip including, an arm, including a tab, a plate, and a grip, the tab including a keyhole aperture, and a biasing member coupled to the plate and extending annularly about an outer surface of the sheath, and configured to bias the tab towards the second configuration, the elongate medical device extending through first keyhole aperture, the grip extending through the sheath aperture.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1B illustrate an exploded view of an embodiment of an intraosseous access system, wherein an access assembly subset of the system is depicted slightly enlarged and in elevation, and an automated driver component is depicted in perspective, in accordance with some embodiments.

FIG. 2A illustrates a cross-section view of a safety mechanism including a clip and a sheath in a first configuration, in accordance with some embodiments.

FIG. 2B illustrates a cross-section view of a safety mechanism including a clip and a sheath in a second configuration, in accordance with some embodiments.

FIG. 3 illustrates a clip of the safety mechanism of FIGS. 2A-2B in an unfolded configuration, in accordance with some embodiments.

FIG. 4A illustrates a side view of the clip of FIG. 3 in a folded configuration, in accordance with some embodiments.

FIG. 4B illustrates a plan view of the clip of FIG. 3 in a folded configuration, in accordance with some embodiments.

FIG. 5A illustrates a plan view of a first tab and a second tab of a clip in a first configuration, in accordance with some embodiments.

FIG. 5B illustrates a plan view of a first tab and a second tab of a clip in a second configuration, in accordance with some embodiments.

FIG. 5C illustrates a side view of a clip and an obturator in a first configuration, in accordance with some embodiments.

FIG. 5D illustrates a side view of a clip and an obturator in a second configuration, in accordance with some embodiments.

FIG. 6A illustrates a clip having a single tab in a first configuration, in accordance with some embodiments.

FIG. 6B illustrates a clip having a single tab in a second configuration, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal-end portion, or the proximal length of the needle can include the proximal end of the needle; however, the proximal portion, the proximal-end portion, or the proximal length of the needle need not include the proximal end of the needle. That is, unless context suggests otherwise, the proximal portion, the proximal-end portion, or the proximal length of the needle is not a terminal portion or terminal length of the needle.

With respect to “distal,” a “distal portion” or a “distal-end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal-end portion, or the distal length of the needle can include the distal end of the needle; however, the distal portion, the distal-end portion, or the distal length of the needle need not include the distal end of the needle. That is, unless context suggests otherwise, the distal portion, the distal-end portion, or the distal length of the needle is not a terminal portion or terminal length of the needle.

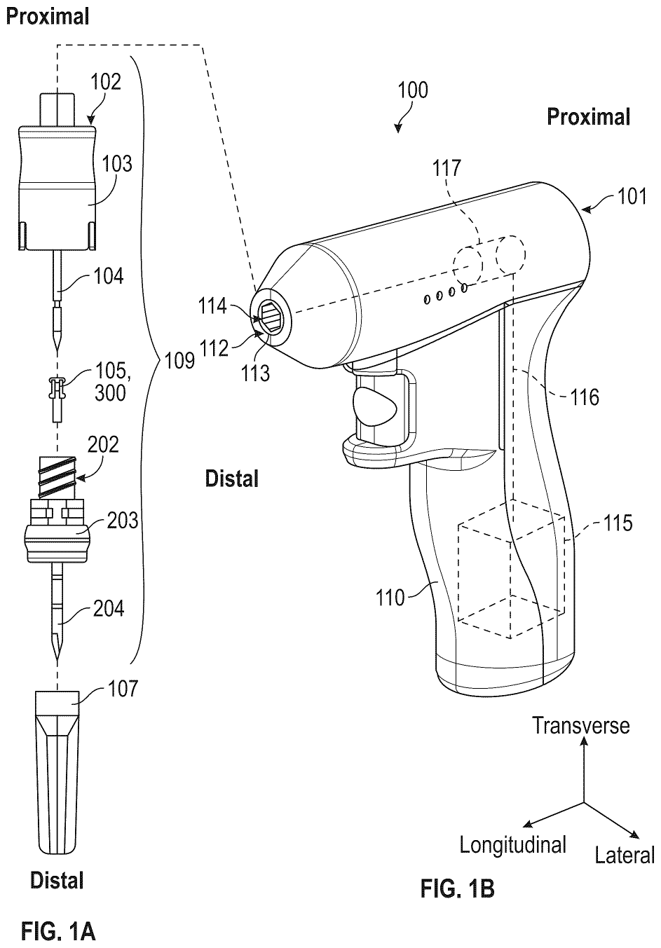

As shown in FIG. 1, and to assist in the description of embodiments described herein, a longitudinal axis extends substantially parallel to an axial length of a needle 204 extending from the driver 101. A lateral axis extends normal to the longitudinal axis, and a transverse axis extends normal to both the longitudinal and lateral axes.

As used herein, the term “spring” is considered to include any type of spring or biasing member that may store potential mechanical energy. Exemplary biasing members can include compression springs, extension springs, torsion springs, constant force springs, flat spring, flexible members, rubber rings, rubber band, leaf spring, V-spring, cantilever spring, volute spring, Belleville spring, gas spring, gravity-propelled biasing members, combinations thereof or the like, and are considered to fall within the scope of the present invention.

Unless defined otherwise, all technical and scientific terms used herein have the same meaning as commonly understood by those of ordinary skill in the art. Although the exemplary embodiments disclosed herein are described with respect to intraosseous (IO) access devices, systems, and methods thereof, it will be appreciated that embodiments disclosed herein can be used with various systems that require capturing a sharp tip of an elongate device to mitigate accidental stick injuries, without limitation.

FIGS. 1A-1B show an exemplary environment of use for a safety mechanism 300, as described herein. FIGS. 1A-1B show an exploded view of an exemplary intraosseous access system (“system”) 100, with some components thereof shown in elevation (FIG. 1A) and other components shown in perspective (FIG. 1B). In an embodiment, the intraosseous access system 100 can be used to penetrate the skin tissues and underlying hard bone (“bone cortex”) for intraosseous access, such as, for example to access the marrow of the bone and/or a vasculature of the patient via a pathway through an interior of the bone (“medullary cavity”).

In an embodiment, the system 100 includes a driver 101 and an access assembly 109. The driver 101 can be used to rotate the access assembly 109 and “drill” a needle 204 into the bone of a patient. In embodiments, the driver 101 can be automated or manual. As shown, the driver 101 is an automated driver 101. For example, the automated driver 101 can be a drill that achieves high rotational speeds. In an embodiment, the intraosseous access system 100 can further include an obturator assembly 102, a safety mechanism or shield 105, and a needle assembly 202, which may be referred to, collectively, as the access assembly 109. The needle assembly 202 can include an access needle (“needle”) 204 supported by a needle hub 203, as described in more detail herein. In an embodiment, the obturator assembly 102 includes an obturator 104. However, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, the obturator assembly 102 may be referred to more generally as an elongated medical device assembly. In like manner, the obturator 104 may be referred to more generally as an elongated medical device.

In an embodiment, the obturator assembly 102 includes a coupling hub 103 that is attached to the obturator 104 in any suitable manner (e.g., one or more adhesives or overmolding). The coupling hub 103 can be configured to interface with the driver 101, as further discussed below. The coupling hub 103 may alternatively be referred to as an obturator hub 103 or, more generally, as an elongated instrument hub 103. In an embodiment, the safety mechanism 105 is configured to couple with the obturator 104 to prevent accidental needle stick injuries when the obturator is removed after placement of the needle 204, as described in more detail herein.

In an embodiment, the needle assembly 202 includes a needle 204. However, in some embodiments, the needle 204 may be replaced with a different instrument, such as, for example, a cannula, a tube, or a sheath, and/or may be referred to by a different name, such as one or more of the foregoing examples. Accordingly, the needle assembly 202 may be referred to more generally as a cannula assembly or as a tube assembly. In like manner, the needle 204 may be referred to more generally as a cannula.

In an embodiment, the needle assembly 202 includes a needle hub 203 that is attached to the needle 204 in any suitable manner. The needle hub 203 can be configured to couple with the obturator hub 103 and may thereby be coupled with the driver 101. The needle hub 203 may alternatively be referred to as a cannula hub 203. In an embodiment, a cap 107 may be provided to cover at least a distal portion of the needle 204 and the obturator 104 prior to use of the access assembly 109. For example, in an embodiment, a proximal end of the cap 107 can be coupled to the obturator hub 103.

With continued reference to FIG. 1B, the driver 101 may take any suitable form. The driver 101 may include a handle 110 that may be gripped by a single hand of a user. In an embodiment, the driver 101 further includes a coupling interface 112, which is formed as a socket 113 that defines a cavity 114. The coupling interface 112 can be configured to couple with the obturator hub 103. In an embodiment, the socket 113 includes sidewalls that substantially define a hexagonal cavity into which a hexagonal protrusion of the obturator hub 103 can be received. Other suitable connection interfaces are also contemplated.

The driver 101 can include an energy source 115 of any suitable variety that is configured to energize the rotational movement of the coupling interface 112. For example, in some embodiments, the energy source 115 may comprise one or more batteries that provide electrical power for the driver 101. In some embodiments, the energy source 115 can comprise one or more springs (e.g., a coiled spring, flat spring, or the like) or other biasing member that may store potential mechanical energy that may be released upon actuation of the driver 101.

The energy source 115 may be coupled with the coupling interface 112 in any suitable manner. For example, in an embodiment, the driver 101 includes an electrical, mechanical, or electromechanical coupling 116 to a gear assembly 117. In some embodiments, the coupling 116 may include an electrical motor that generates mechanical movement from electrical energy provided by an electrical energy source 115. In other embodiments, the coupling 116 may include a mechanical linkage to the gear assembly 117. The driver 101 can include a mechanical coupling of any suitable variety to couple the gear assembly 117 with the coupling interface 112. In other embodiments, the gear assembly 117 may be omitted.

Further details and embodiments of the intraosseous access system 100 can be found in WO 2018/075694, WO 2018/165334, WO 2018/165339, and U.S. Pat. No. 10,893,887, each of which are incorporated by reference in their entirety into this application.

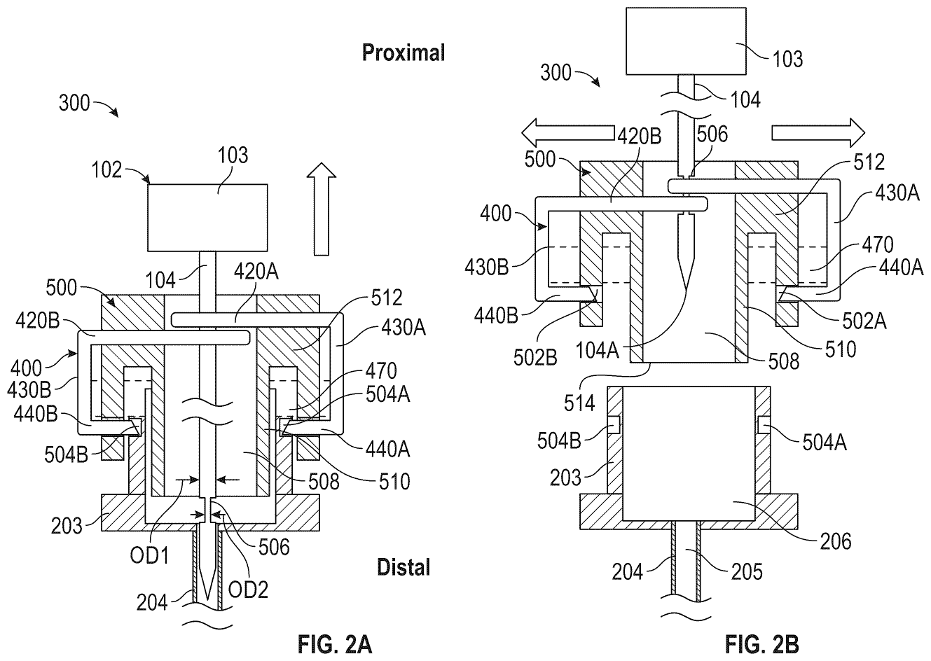

FIGS. 2A-5D illustrate various details of an embodiment of a safety mechanism 300. The safety mechanism 300 can be used with an intraossesous access system 100, as described herein, for example, the safety mechanism 300 can be used in place of the safety shield 105. However, it will be appreciated that embodiments of the safety mechanism 300 can be used with various needles, cannulas, trocars, stylets, obturators, or similar sharpened medical devices without limitation, and can be configured to prevent accidental needle-stick injuries.

As shown in FIGS. 2A-2B, in an embodiment, the safety mechanism 300 can generally include a clip 400 and a sheath 500. A portion of the clip 400 can slidably engage the sheath 500 and transition between a first, or unlocked, configuration (FIG. 2A) and a second, or locked, configuration (FIG. 2B). In the first configuration, as shown in FIG. 2A, the clip 400 can slidably engage a shaft of the obturator 104 along a longitudinal axis thereof. Further, the clip 400 can also selectively engage the needle hub 203 and secure the sheath 500 thereto, preventing any relative longitudinal movement between the sheath 500 and the needle hub 203.

As the obturator 104 is withdrawn proximally from the needle 204 and slides relative to the safety mechanism 300, an annular notch (“notch”) 506 aligns with a portion of the clip 400, allowing the clip 400 to transition from the first configuration (FIG. 2A) to the second configuration (FIG. 2B). In the second configuration, the clip 400 is locked to the obturator 104, preventing any relative longitudinal movement between the obturator 104 and safety mechanism 300. Further, the clip 400 disengages the needle hub 203 allowing the obturator 104, clip 400, and sheath 500 assembly to separate from the needle hub 203.

Advantageously, the clip 400 maintains the sheath 500 in a longitudinally fixed position relative to the tip of the obturator 104 preventing accidental stick injuries. Further, the safety mechanism 300 provides a simplified manufacturing process, and associated cost savings. Advantageously, the clip 400 requires only a relatively small surface area to contact the obturator 104, reducing friction or “drag” therebetween as the obturator 104 is slid proximally. Further the edges of the clip 400 can be smoothed and/or beveled to further reduce the friction between the clip 400 and the obturator 104. As such, less force is required to remove the obturator 104 from the needle 204 and can prevent the obturator 104 from becoming prematurely detached from the driver 101. Advantageously, the engagement between the clip 400, the sheath 500, and the needle hub 203 in the first configuration (FIG. 2A) requires much higher override forces providing a much more robust interface, mitigating accidental failure of the safety mechanism 300.

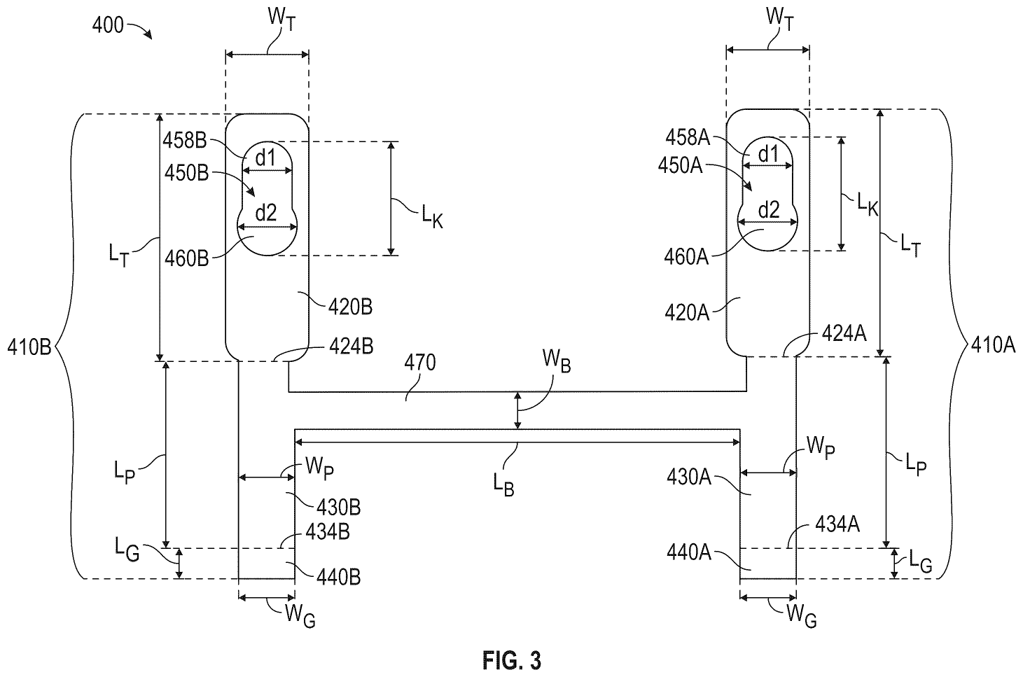

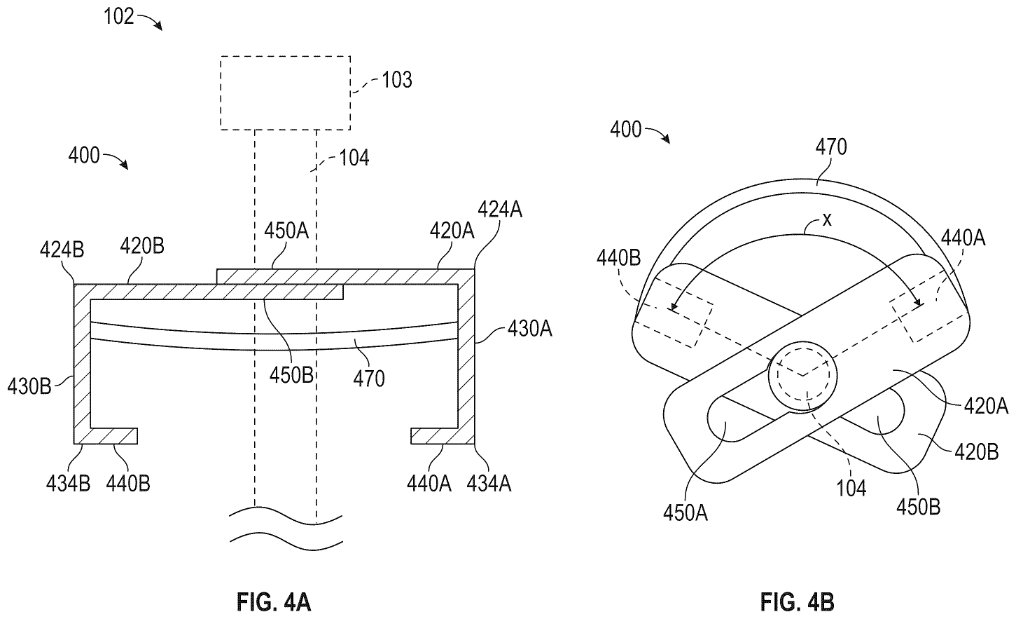

FIGS. 3-4B show further details of the clip 400 of the safety mechanism 300. FIG. 3 shows the clip 400 in an unfolded configuration. FIGS. 4A-4B show the clip 400 in a folded configuration. In an embodiment, the clip 400 can be formed as a single monolithic piece. For example, the safety mechanism can be stamped from a sheet of metal, polymer, plastic, composite, or the like and shaped into the folded configuration (FIGS. 4A-4B). Optionally, a portion of the clip 400 can be heated to facilitate folding of the clip 400. Advantageously, the clip 400, formed as such, simplifies the manufacturing process and reduces associated costs and complexity. FIG. 4A shows a side view of the clip 400 in a folded configuration. FIG. 4B shows a plan view of the clip 400 in a folded configuration.

The clip 400 generally includes a first arm 410A and a second arm 410B and a biasing member 470 extending therebetween. In an embodiment, each arm 410 can include a tab 420 (e.g. a first tab 420A and a second tab 420B), a plate 430 (e.g. a first plate 430A and a second plate 430B), and a grip 440 (e.g. a first grip 440A and a second grip 440B). In an embodiment, the biasing member 470 can be a flat spring, a leaf spring, or the like, and can extend between the first plate 430A and the second plate 430B. In an embodiment, the biasing member 470 can extend between the first tab 420A and the second tab 420B.

As shown in FIG. 3, each arm 420 can include a tab foldline 424, disposed between the tab 420 and the plate 430, and a grip foldline 434 disposed between the plate 430 and the grip 440. In an embodiment, the tab foldline 424 or the grip foldline 434 can include a score line, groove, perforation, laser cut line or similar line of weakness to facilitate folding of the arm 410 therealong, as described in more detail herein.

In an embodiment, each tab 420 can define a tab length (L_{T}) and a tab width (W_{T}). Each plate 430 can define a plate length (L_{P}) and a plate width (W_{P}). Each grip 440 can define a grip length (L_{G}) and a grip width (W_{G}). In an embodiment, the tab length (L_{T}) can be equal to, or greater than, the tab width (W_{T}). In an embodiment, the tab length (L_{T}) can be equal to, or greater than the plate length (L_{P}). In an embodiment, the tab width (W_{T}) can be equal to, or greater than the plate width (W_{P}).

In an embodiment, the plate length (L_{P}) can be equal to or greater than the plate width (W_{P}). In an embodiment, the plate length (L_{P}) can be equal to, or greater than the grip length (L_{G}). In an embodiment, the grip length (L_{G}) can be equal to, less than, or greater than the grip width (W_{G}). In an embodiment, the grip width (W_{G}) can be substantially equal to the plate width (W_{P}). In an embodiment, the grip width (W_{G}) can be greater than, or less than the plate width (W_{P}).

In some embodiments, the tab length (L_{T}) of the first tab 420A can be equal to the tab length (L_{T}) of the second tab 420B. In some embodiments, the tab length (L_{T}) of the first tab 420A can be different from the tab length (L_{T}) of the second tab 420B. In some embodiments, the plate length (L_{P}) of the first plate 430A can be equal to the plate length (L_{P}) of the second plate 430B. In some embodiments, the plate length (L_{P}) of the first plate 430A can be different from the plate length (L_{P}) of the second plate 430B. In some embodiments, the grip length (L_{G}) of the first grip 440A can be equal to the grip length (L_{G}) of the second grip 440B. In some embodiments, the grip length (L_{G}) of the first grip 440A can be different from the grip length (L_{G}) of the second grip 440B.

In an embodiment, the tab 420 can further include a keyhole aperture 450 substantially defining a “keyhole” shape and defining a keyhole aperture length (L_{K}). For example, the first tab 420A can include a first keyhole aperture 450A, and the second tab 420B can include a second keyhole aperture 450B. Each keyhole aperture 450 can include a lock aperture 458 communicating with an unlock aperture 460. The lock aperture 458 can define a first inner diameter (d1), and the unlock aperture 460 can define a second inner diameter (d2), larger than the first diameter (d1). In an embodiment, the diameter (d2) of the unlock aperture 460 is equal to or greater than an outer diameter of the obturator 104.

In an embodiment, the diameter (d1) of the lock aperture 458 is less than an outer diameter of the obturator 104. In an embodiment, the diameter (d1) of the lock aperture 458 is equal to or greater than the diameter of the notch 506. To note, the outer diameter (OD2) of the notch 506 is less than an outer diameter (OD1) of the obturator 104. In an embodiment, the keyhole aperture length (L_{K}) can be equal to or greater than the first diameter (d1) and the second diameter (d2) combined.

In an embodiment, the biasing member 470 can extend from the first arm 410A to the second arm 410B to define a biasing member length (L_{B}), and can define a biasing member width (W_{B}) extending perpendicular thereto. In an embodiment, the biasing member length (L_{B}) is greater than the biasing member width (W_{B}). In an embodiment, as shown in FIG. 4B, the biasing member length (L_{B}) can extend through less than 360° of the circumference of the sheath 500, e.g. through an arc distance (x).

In an embodiment, the biasing member 470 can couple to the first arm 410A or the second arm 410B at any position therealong. In an embodiment, the biasing member 470 can couple to the first plate 430A or the second plate 430B at any position therealong. In an embodiment, an axis of the elongate biasing member 470 can extend perpendicular to an axis of the first arm 410A or the second arm 410B and can couple to the first arm 410A and the second arm 410B at a similar position therealong. In an embodiment, an axis of the elongate biasing member 470 can extend at an angle relative to an axis of the first plate arm 410A or the second arm 410B and can couple to the first arm 410A and the second arm 410B at different positions.

In an embodiment, the keyhole apertures 450 can be oriented on the respective tabs 420 in the same direction or in different directions. For example, the first keyhole aperture 450A can be oriented with the first unlock 460A aperture disposed closer to the first plate 430A and the second keyhole aperture 450B can be oriented with the second lock aperture 458B disposed closer to the second plate 430B. In other embodiments, the first keyhole aperture 450A can be oriented with the first lock aperture 458A disposed closer to the first plate 430A and the second keyhole aperture 450B can be oriented with the second unlock aperture 460B disposed closer to the second plate 430B. These and other orientations of keyhole apertures are considered to fall within the scope of the present invention.

FIG. 4A shows a side view of the clip 400 in the folded configuration. FIG. 4B shows a plan view of the clip 400 in the folded configuration. In an embodiment, to transition the clip 400 from the unfolded configuration (FIG. 3) to the folded configuration (FIGS. 4A, 4B), the first tab 420A can be folded, relative to the first plate 430A, along the first plate fold line 424A, through an angle of substantially 90°. The second tab 420B can be folded, relative to the second plate 430B, along the second plate fold line 424B, through an angle of substantially 90°. The first grip 440A can be folded, relative to the first plate 430A, along the first grip fold line 434A through an angle of substantially 90°. The first grip 440A can extend substantially parallel to the first tab 420A. The second grip 420B can be folded, relative to the second plate 430B, along the second grip fold line 434B, through an angle of substantially 90°. The second grip 440B can extend substantially parallel to the second tab 420B.

The elongate biasing member 470 can be elastically deformed such that a portion of first keyhole aperture 450A overlaps a portion of the second keyhole aperture 450B and aligns with a central longitudinal axis, substantially defined by obturator 104. As such, in the folded configuration, the first tab 420A and the second tab 420B extend radially inward. Similarly, the first grip 440A and the second grip 440B extend radially inward, relative to a central longitudinal axis of the obturator 104. The first plate 430A and the second plate 430B can extend substantially parallel to the central longitudinal axis.

In the folded configuration, in an embodiment, the first tab 420A can be positioned above the second tab 420B. In other embodiments, the second tab 420B can be positioned above the first tab 420A. In some embodiments, the elongated biasing member 470 can be elastically deformed to define an arcuate shape. The biasing member 470 can extend along a portion of the circumference of the sheath 500. As shown in FIG. 4B, in an embodiment, the elongate biasing member 470, in the elastically deformed configuration, extends through an arc distance (x) of between 5° and 360°. In an embodiment, the elongate biasing member 470 in the elastically deformed configuration extends through an arc distance of between 100° and 200°.

In an embodiment, the clip 400 may be formed by 3D printed, injection molded, or the like, in the folded configuration, or formed in the unfolded configuration and then folded to the folded configuration, as described herein. In an embodiment, the clip 400 can be formed from two or more pieces joined together by welding, bonding, adhesive, or the like in the folded configuration, or formed in the unfolded configuration and then folded to the folded configuration, as described herein.

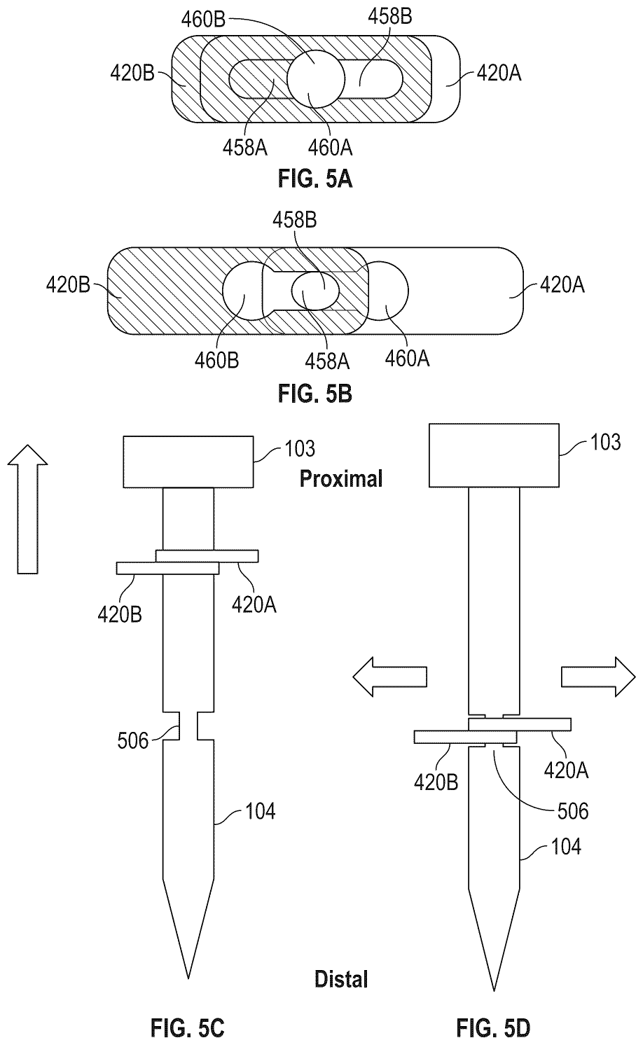

FIGS. 5A-5D show an exemplary method of use for the clip 400. FIG. 5A shows a plan view of the first tab 420A and the second tab 420B in the first, or unlocked, configuration. FIG. 5B shows a plan view of the first tab 420A and the second tab 420B in the second, or locked, configuration. FIG. 5C shows a side view of the obturator 104 and the first tab 420A and the second tab 420B in the first configuration. FIG. 5D shows a side view of the obturator 104 and the first tab 420A and the second tab 420B in the second configuration. To note, the biasing member 470 is not shown for clarity. Further, the first tab 420A is shown as ghosted to show the relative position of the second tab 420B disposed there below. The elastic properties of the elongate biasing member 470 biases the first tab 420A and the second tab 420B towards the second configuration (FIG. 5B).

In the first configuration, as shown in FIGS. 5A and 5C, the first unlock aperture 460A aligns with the second unlock aperture 460B to define an opening sufficient to allow an outer diameter of the obturator 104 to pass therethrough. In some embodiments, the first configuration with the first unlock aperture 460A and the second unlock aperture 460B aligning is the natural higher energy state of the clip 400. As illustrated in FIGS. 5B and 5D, as the obturator 104 is withdrawn proximally from the needle 204, the notch 506 of the obturator 104 longitudinally aligns with the first keyhole aperture 450A and the second keyhole aperture 450B. Since the outer diameter (OD2) of the notch 506 is less than the diameter (d1) of the lock aperture 458, the notch 506 allows the biasing member 470 to transition the clip 400 from the first configuration to the second configuration. In the second configuration, the first tab 420A and the second tab 420B are displaced radially outward relative to a central longitudinal axis.

In some embodiments, the second configuration with the first lock aperture 458A and the second lock aperture 458B aligning with the central longitudinal axis, is the natural lower energy state of the clip 400. To note, as shown in FIGS. 5B and 5D, the first tab 420A, with the first grip 440A coupled thereto, and the second tab 420B, with the second grip 440B coupled thereto, are disposed radially outward in the second configuration, relative to the first configuration.

Advantageously, the clip 400 in the second configuration mitigates failure of the safety mechanism 300. The biasing member 470 urges the first tab 420A and the second tab 420B to engage the notch 506 and prevent vertical movement of the obturator 104 relative to the clip 400. As such, to transition the clip 300 from the second configuration to the first configuration and allow relative vertical movement of the obturator 104, the notch 506 must be longitudinally aligned with the keyhole apertures 450A. The first arm 410A and the second arm 410B must be urged radially inward, perpendicular to the longitudinal axis, and with sufficient force to overcome the force of the biasing member 470. The first unlock aperture 460A and the second unlock aperture 460B must be displaced radially inward and aligned with the central longitudinal axis. Advantageously, applying an axial force alone cannot cause the clip 400 to accidentally transition from the second configuration to the first configuration, causing accidental disengagement of the sheath 500 from the tip of the obturator 104.

With continued reference to FIGS. 2A-2B, the safety mechanism 300, including the clip 400 and the sheath 500 can selectively engage the needle hub 203. FIG. 2A shows the safety mechanism 300 in the first configuration, whereby the obturator 104 is slidably engaged with the clip 400. FIG. 2B shows the clip 400 in the second configuration whereby the sheath 500 is in a fixed longitudinal position relative to a distal tip of the obturator 104. With the safety mechanism 300 in the second configuration, a distal edge 514 of the sheath 500 extends distally of the distal tip 104A of the obturator 104 to mitigate accidental stick injuries.

The needle hub 203 can support the needle 204 defining a needle lumen 205. The needle hub 203 can define a hub recess 206 communicating with the needle lumen 205. The sheath 500 can include a body 510, defining a sheath lumen 508, and a shroud 512 extending axially therefrom. A portion of the sheath body 510 can be configured to extend into the needle hub recess 206 and can engage an inner surface thereof. In an embodiment, the sheath 500, or portion thereof (e.g. the body 510 or the shroud 512), can engage the needle hub 203 in an interference fit, snap-fit engagement, luer lock engagement, threaded engagement, combinations thereof, or the like.

The sheath lumen 508 can be configured to receive the obturator 104 extending therethrough. The sheath 500 can further include a shroud 512 extending axially. The sheath 512 can extend over an outer surface of the needle hub 203 when the sheath body 510 is engaged with the needle hub recess 206. In an embodiment, an inner surface of the shroud 512 can slidably engage an outer surface of the needle hub 203. In an embodiment, the sheath shroud 512 can include one or more sheath apertures 502, e.g. a first aperture 502A and a second aperture 502B, extending through the shroud 512, perpendicular to the longitudinal axis. In some embodiments, the one or more apertures 502 can communicate with a channel (not shown) extending annularly around the outer circumference of the sheath shroud 512.

In the first configuration (FIG. 2A), the first arm 410A and the second arm 410B are disposed radially inward relative to each other and the obturator 104 can slide longitudinally relative to the safety mechanism 300. The grip 440 of the arm 410 can extend through the aperture 502 and engage an outer surface of the needle hub 203. In an embodiment, the grip 440 can engage a grip recess 504 disposed in an outer surface of the needle hub 203. For example, as shown in FIG. 2A with the sheath 500 engaged with the needle hub 203, a first grip 440A can extend through the first sheath aperture 502A and engage a first grip recess 504A. A second grip 440B can extend through the second sheath aperture 502B and engage a second grip recess 504B. As such, the clip 400 can secure the sheath 500 to the needle hub 203 and can prevent disengagement therefrom when an axial force is applied.

Advantageously, the sheer strength of the grips 440 mitigates premature disengagement of the safety mechanism 300 from the needle hub 203 as the obturator 104 is withdrawn proximally. Worded differently, the sheer forces of the grip 440 interacting with the grip recess 504 are greater than the frictional forces of the keyhole apertures 450 interacting with the obturator 104. As such, the grip 440 prevents the sheath 500 from disengaging the needle hub 203.

As shown in FIG. 2B, as the obturator 104 is withdrawn proximally, the notch 506 longitudinally aligns with the keyhole apertures 450A, 450B. The smaller outer diameter of the notch 506 can allow the clip 400 to transition from the first configuration to the second configuration. In the second configuration the first arm 410A and the second arm 410B can be displaced radially outward relative to each other. As such the grips 440A, 440B are also displaced radially outward and disengage grip recesses 504A, 504B of the needle hub 203. As such, the sheath 500 can disengage the needle hub 203. In an embodiment, the length (L_{G}) of the grips 440A, 440B, are sufficient to allow the grips 440A, 440B to remain engaged with the sheath apertures 502A, 502B respectively, so that the clip 400 remains engaged with the sheath 500 in both the first configuration and the second configuration. The obturator 104, with the safety mechanism 300 engaged there can be removed. As noted, the distal tip 514 of the sheath 500 in the second configuration extends distally of the distal tip 104A of the obturator 104 to prevent accidental needle stick injuries.

Advantageously, embodiments of the safety mechanism 300, as described herein, provide a simplified manufacturing process and reduce associated costs. The clip 400 can be stamped from a single sheet of material and folded into position. The sheath 500 and needle hub 203 can be molded in a single process that negates any secondary machining.

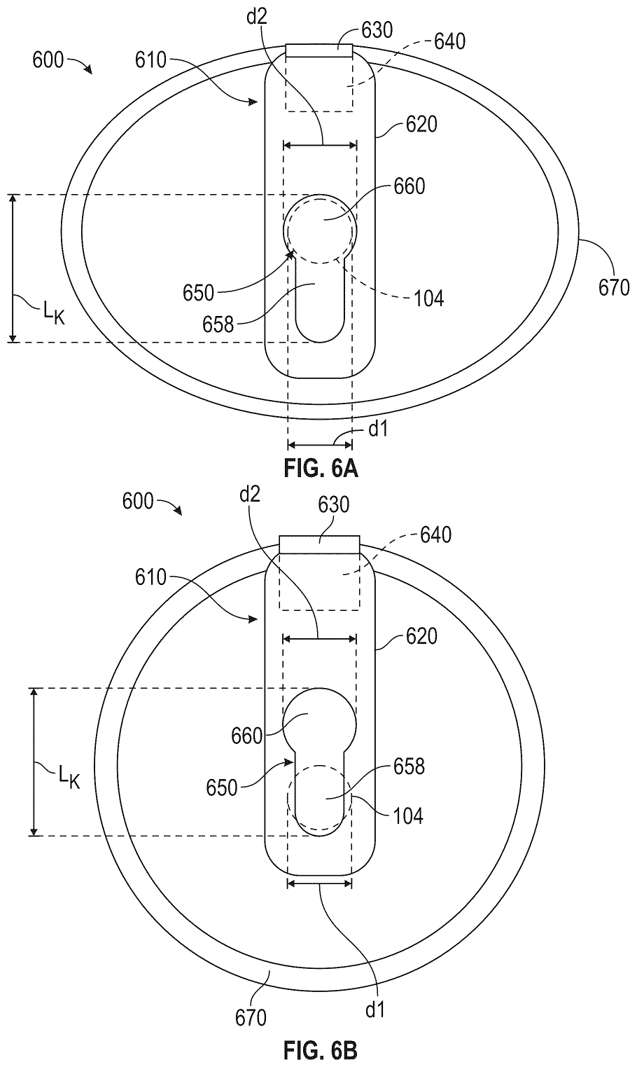

FIGS. 6A-6B show an embodiment of a clip 600 including a single tab 620 configured to be used with the sheath 500 of the safety mechanism 300. FIG. 6A shows a plan view of the clip 600 in the first configuration. FIG. 6B shows a plan view of the clip 600 in the second configuration. The clip 600 can include an arm 610 and a biasing member 670 coupled thereto. In an embodiment, the arm 610 can include a tab 620, a plate 630 and a grip 640. The tab 620 includes a keyhole aperture 650 defining a keyhole aperture length (L_{K}) and having a lock aperture 658 communicating with an unlock aperture 660.

In an embodiment, the biasing member 670 extending annularly about the sheath 500. For example, the biasing member 670 can be coupled to the plate 630 and extend through 360° around the entire outer circumference of the sheath 500. In an embodiment, the biasing member 670 can extend through less than 360° e.g. around at least half of the outer circumference of the sheath 500. In an embodiment, a portion of the biasing member 670, e.g. a portion disposed opposite the plate 630 can be coupled with sheath 500. In an embodiment, the elongate biasing member 670 can elastically deform between a first configuration (FIG. 6A), and a second configuration (FIG. 6B). The biasing member 670 biases the clip 600 towards a second configuration (FIG. 6B). In the first configuration, the grip 640 can engage a sheath aperture 502 and a grip recess 504 to engage the sheath 500 with the needle hub 203, as described herein. In the second configuration, the tab 620 engages the notch 506 of the obturator 104 to lock the sheath 500 relative to the obturator tip 104A, the radially outward movement of the plate 630 disengages the grip 640 from the grip recess 504 allowing the safety mechanism 300 to disengage the needle hub 203, as described herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A safety mechanism for an obturator having a sharp distal tip and a notch proximal to the sharp distal tip, the safety mechanism comprising:
a sheath and clip combination configured to releasably couple to a needle hub, the clip configured to transition between a coupled configuration and an uncoupled configuration, the clip comprising:
a first arm including a first keyhole aperture;
a second arm including a second keyhole aperture; and
a biasing member connecting the first arm to the second arm, the biasing member configured to bias the clip toward the uncoupled configuration, wherein:
the first keyhole aperture and the second keyhole aperture are configured to slidingly receive the obturator, and
the clip transitions from the coupled configuration to the uncoupled configuration when the first keyhole aperture and the second keyhole aperture engage the notch.

2. The safety mechanism according to claim 1, wherein the first arm and the second arm are spaced in a radially outward position in the uncoupled configuration relative to the coupled configuration.

3. The safety mechanism according to claim 1, wherein the first keyhole aperture includes a first lock aperture communicating with a first unlock aperture, and the second keyhole aperture includes a second lock aperture communicating with a second unlock aperture, one or both of the first lock aperture and the second lock aperture having a first inner diameter, and one or both of the first unlock aperture and the second unlock aperture having a second inner diameter larger than the first inner diameter.

4. The safety mechanism according to claim 3, wherein the first inner diameter is less than the first outer diameter and equal to or larger than the second outer diameter, and the second inner diameter is equal to or larger than the first outer diameter.

5. The safety mechanism according to claim 3, wherein the first lock aperture and the second lock aperture are axially aligned when the clip is in the uncoupled configuration, and the first unlock aperture and the second unlock aperture are axially aligned when the clip is in the coupled configuration.

6. The safety mechanism according to claim 3, wherein the first unlock aperture and the second unlock aperture are spaced radially outward relative to a central axial position in the uncoupled configuration.

7. The safety mechanism according to claim 3, wherein the first lock aperture is disposed opposite the longitudinal axis from the second lock aperture in the coupled configuration.

8. The safety mechanism according to claim 1, wherein the sheath includes a body portion configured for insertion into a proximal end of the needle hub and a shroud configured to extend over an outer surface of the needle hub in the coupled configuration.

9. The safety mechanism according to claim 8, wherein the needle hub includes a first needle hub recess, wherein the shroud includes a first shroud aperture, and wherein the clip includes a first grip portion extending through the first shroud aperture and into the first needle hub recess in the coupled configuration.

10. The safety mechanism according to claim 9, wherein the needle hub includes a second needle hub recess, wherein the shroud includes a second shroud aperture, and wherein the clip includes a second grip portion extending through the second shroud aperture and into the second needle hub recess in the coupled configuration.

11. The safety mechanism according to claim 9, wherein the first grip portion disengages from the first needle hub recess in the uncoupled configuration.

12. The safety mechanism according to claim 10, wherein the first grip portion and the second grip portion disengage from the first needle hub recess and the second needle hub recess, respectively, in the uncoupled configuration.

13. The safety mechanism according to claim 12, wherein the first grip portion and the second grip portion remain in the first shroud aperture and second shroud aperture, respectively, in the uncoupled configuration.

14. The safety mechanism according to claim 1, wherein the clip is formed as a monolithic piece of material from one of a plastic, a polymer, a metal, an alloy, or a composite.
